(19) United States
(12) Patent Application Publication
(10) Pub. No.: US 2007/0141612 A1
(43) Pub. Date: Jun. 21, 2007

(54) Systematic in silico selection method for identifying drug targets in pathogens

(75) Inventor: Sanjay Kumar, Ipswich, MA (US)

Correspondence Address: HARRIET M. STRIMPEL; NEW ENGLAND BIOLABS, INC., 240 COUNTY ROAD, IPSWICH, MA, 01938-2723, US

(73) Assignee: New England Biolabs, Inc., Ipswich, MA (US)

(21) Appl. No.: 11/639,025

(22) Filed: Dec. 14, 2006

Related U.S. Application Data
(60) Provisional application No. 60/751,396, filed on Dec. 16, 2005.

(51) Int. Cl. C12Q 1/68; G01N 33/53; G06F 19/00

(52) U.S. Cl. 435/6; 435/7.1; 702/20

(57) ABSTRACT

Methods and compositions are provided for selecting drug targets in silico, that include the following steps performed in the order presented or an alternative order of partially or entirely at the same time: (a) identifying one or more essential or functionally important sequences from a model organism using pre-existing genomic and phenotypic data; (b) comparing sequences from (a) with a DNA or peptide sequence from a pathogen to store homologous sequences; (c) comparing sequences from the pathogen with the DNA or peptide sequence from a host organism to store those sequences absent in the host organism; and (d) comparing sequences from (b) and (c) to identify shared sequences, the shared sequences being a drug target. Additionally, identified drug targets are provided.

### CROSS REFERENCE

This application claims priority from U.S. Provisional Application Ser. No. 60/751,396 filed Dec. 16, 2005.

### BACKGROUND

Pathogenic organisms whether prokaryotic or eukaryotic infect host organisms to cause disease. It is desirable to treat the diseased host with a therapeutic agent or drug that is toxic for the pathogen but leaves the host unharmed. Unfortunately, finding suitable drug targets in the pathogen is frequently problematic because of the lack of knowledge about the pathogen's biology.

### DETAILED DESCRIPTION OF THE EMBODIMENTS

A method is provided for finding drug targets by an approach that utilizes a bioinformatics approach. This method requires selecting a suitable model organism so as to generate a set of essential genes or gene products, which are likely to be shared by the pathogen. The choice of the model organism may vary over time according to availability of genomic sequences and phenotypic data in the database. The closer in evolutionary terms the model organism is to the pathogen, the more likely a subset of essential genes will be found to be shared between both the model and pathogenic organisms but will be absent in an evolutionarily distant host genome. Consequently, in one embodiment of the invention, the computer is capable of identifying the phylogenetically closest model organism to the pathogen for which an existing database contains an amount of genome sequence and phenotype data above an arbitrarily assigned threshold. Having selected the model organism using the above criteria, the computer searches the databases to identify a set of genomic sequences that encode essential or functionally important proteins. The essential nature of the protein is defined by the impact of its phenotype on the viability of the pathogen. The model organism can be virtual including genomic data and phenotypic data compiled in silico from a number of closely related real organisms. In the example provided herein, i C. elegans is selected as a model organism for pathogenic nematodes.

In one embodiment of the invention, a computer-based system is provided for identifying drug targets that includes: (a) a memory for storing a plurality of databases, wherein the plurality of databases comprise a plurality of sequence databases; (b) a processor in communication with the memory; and (c) an output device in communication with the processor, wherein the processor is configured to: (i) group sequences belonging to a model organism, a pathogen and a host from one or more of the plurality of sequence databases; (ii) identify essential or functionally important sequences from the model organism by matching phenotypic data with sequences of the model organism grouped in (i); (iii) compare sequences of the pathogen with sequences identified in (ii); (iv) compare sequences of the pathogen with the host sequences and select pathogen sequences that do not share sequence similarity with the host sequences; (v) compare sequences from (iii) and (iv) to identify sequences corresponding to sequences encoding drug targets; and (vi) cause at least some of the sequences identified in (v) to be displayed on the output device. The above steps can be performed in any order or partially or entirely at the same time.

In an embodiment of the invention, the method for identifying drug targets includes one or more of the following steps:

1. Group sequences belonging to the model organism in Database I.

2. Identify essential or functionally important sequences by matching phenotypic data with sequences in Database I and store these selected sequences in Database II.

3. Group pathogen sequences in Database III.

4. Group host sequences in Database IV.

5. Compare sequences from Database III with those in Database II and store sequences that are significantly similar to those in Database II in Database V.

6. Compare sequences from Database III with those in Database IV and store those sequences from Database III that do not have similarity to sequences in Database IV in Database VI.

7. Compare sequences in Database V and VI and store shared sequences in Database VII as encoding or constituting drug targets.

In an embodiment of the invention, the above databases can be constructed from information about genomes of the target pathogen(s) and the target host(s), and phenotypic data relating to the essentiality or functional importance of various sequences (essential model sequences) from a real or virtual model organism representing the pathogen. The latter set of sequences could be pre-existing or may be constructed by systematically evaluating each sequence from a real or virtual model organism as follows (steps 1-4). If a set of essential model sequences is pre-existing, steps 1-4 of the following algorithm may be omitted.

1. Select an arbitrary DNA or peptide sequence from the model organism (model organism sequence) from the database of model organism sequences.

2. Retrieve functional/phenotypic data from auxiliary data sources corresponding to the model organism sequence.

3. If the functional/phenotypic data from step 2 indicates that the model organism sequence is essential or important for normal development or functioning of the model organism, add the sequence to the set of essential model sequences; otherwise, abort and return to step 1.

4. If more model organism sequences remain, return to step 1; otherwise proceed to step 5.

5. Select an arbitrary DNA or peptide sequence from the pathogen (pathogen sequence) from the database of pathogen sequences.

6. Using a comparative sequence analysis method applicable to DNA or peptide sequences, determine if sequences orthologous to the pathogen sequence are present in the genome of the target host and are in the essential or functionally important model organism sequence set.

7. If the results of step 6 indicate that orthologs of the pathogen sequence are absent in the genome of the target host but present in the set of essential or functionally important model sequences, record the identities of the target pathogen in a list of drug targets.

8. If more pathogen sequences are available, return to step 5.

9. Annotate the set of sequences in the list of drug targets using any auxiliary data sources.

10. Prioritize the list of drug targets on the basis of suitability, either manually or programmatically.

The biology of the sequences in the model organism may be determined by gene silencing, gene knockout or other methods known in the art for studying gene function. Step 6 above may be accomplished using a comparative sequence analysis method that allows relatedness determination, for example, a BLAST program (Altschul et al., J. Mol. Biol. 215: 403-410 (1990)) or FASTA (Pearson and Lipman Proc. Natl. Acad. Sci 85:2444-2448 (1988)). Step 10 may use a prioritization protocol based on predictions of a person of ordinary skill in the art with respect to, for example, (a) ability to clone and overexpress DNA (size of gene, isoelectric point etc.); (b) solubility of protein product; (c) availability of an assay for expression product; (d) availability of a protein structure; or (e) impact of phenotype on the viability of the organism e.g., lethality in the embryo or uncoordinated motion in the embryo or disorganization in development of tissue structures, etc. Any of the above parameters may be evaluated by a subjective value structure selected by the experimenter.

### EXAMPLE

### Example 1

### Implementation of the Algorithm to Discover Drug Targets in Brugia Malayi

The target pathogen was Brugia malayi. Databases of genomic DNA sequences as well as sequences predicted to encode protein sequences in Brugia malayi were available from TIGR (The Institute for Genomic Research, USA). The target host was Homo sapiens (human). A database of peptide sequences of human origin was constructed from the public sequence databases available at NCBI (National Center for Biotechnology Information, USA). The model organism for this study was Cenorhabditis elegans. C. elegans peptide sequences and the identities of the genes encoding them were obtained from Wormbase (www.wormbase.org) as wormpep release 150. Phenotypic data for the model organism sequences consisted of the results of genome-wide RNAI knockdown scans of the C. elegans genome. The results were obtained from Wormbase via their WormMart service at the same time that release 150 of wormpep was considered current.

An essential model sequence set was established by retrieving the RNAi phenotype corresponding to the source gene for each peptide in Wormpep. Peptides were included in the essential model sequence set if the corresponding RNAi phenotype was anything other than Wild Type, unclassified, or missing.

Each pathogen sequence was compared to every sequence in the essential model sequence set and target host sequence databases using the BLAST sequence analysis program blastp. A variety of expectation value (e-value) cutoffs were employed in the blastp analysis to tune the stringency of the analysis and limit the size of the drug target list. For the list shown [below], the e-value cutoffs were 1.0×10⁻¹³ and 1.0×10⁻²⁰ for the human and Brugia malayi sequence comparisons, respectively. Pathogen sequences that had no human orthologs with e-values smaller than the cutoff, but did have essential C. elegans orthologs with e-values smaller than the cutoff were placed into the list of drug targets. In the list [below] they are shown with the identifier from the TIGR genome sequence database for the B. malayi genome, along with the contig on which the sequence was located as well as its start and stop coordinates along that contig.

TABLE 1

| | TIGR pub | | | | |
|---|---|---|---|---|---|
| TIGR gene ID | locus | Gene Description | Contig | Start | Stop |
| 12415.m00014 | Bm1_00120 | ATP synthase epsilon | 12415 | 1145 | 966 |
| | | chain, mitochondrial, | | | |
| | | putative | | | |
| 12422.m00027 | Bm1_00215 | conserved hypothetical | 12422 | 2992 | 554 |
| | | protein | | | |
| 12443.m00018 | Bm1_00430 | hypothetical protein | 12443 | 1653 | 1709 |
| 12453.m00017 | Bm1_00490 | CEH-25 homeobox | 12453 | 561 | 2316 |
| | | protein-related | | | |
| 12478.m00033 | Bm1_00640 | hypothetical protein | 12478 | 2057 | 17 |
| 12482.m00020 | Bm1_00705 | P. falciparum RESA-like | 12482 | 57 | 1507 |
| | | protein with DnaJ | | | |
| | | domain-related | | | |
| 12483.m00026 | Bm1_00720 | conserved hypothetical | 12483 | 3719 | 10670 |
| | | protein | | | |
| 12496.m00298 | Bm1_00815 | Cuticle collagen 14, | 12496 | 50733 | 52051 |
| | | putative | | | |
| 12506.m00096 | Bm1_00910 | Zinc finger, C2H2 type | 12506 | 19107 | 22372 |
| | | family protein | | | |
| 12524.m00104 | Bm1_01030 | hypothetical protein | 12524 | 6552 | 7247 |
| 12525.m00011 | Bm1_01060 | Calcium-binding | 12525 | 4849 | 2413 |
| | | protein.-related | | | |
| 12527.m00016 | Bm1_01075 | CG15780-PA-related | 12527 | 180 | 763 |
| 12528.m00040 | Bm1_01100 | hypothetical protein | 12528 | 11394 | 10495 |
| 12582.m00015 | Bm1_01505 | Hypothetical protein | 12582 | 2634 | 2840 |
| 12584.m00063 | Bm1_01555 | hypothetical protein | 12584 | 6119 | 4890 |
| 12601.m00336 | Bm1_01765 | Probable mitochondrial | 12601 | 58355 | 57798 |
| | | import receptor subunit | | | |
| | | TOM7-like | | | |
| 12616.m00134 | Bm1_01935 | Hypothetical protein | 12616 | 49650 | 47926 |
| 12617.m00005 | Bm1_01955 | LPXTG cell wall surface | 12617 | 1877 | 1647 |
| | | anchor family protein, | | | |
| | | putative | | | |
| 12646.m00015 | Bm1_02135 | ribosomal protein L9 | 12646 | 1677 | 2711 |
| | | domain containing | | | |
| | | protein | | | |
| 12647.m00026 | Bm1_02140 | Lethal protein 805, | 12647 | 1694 | 117 |
| | | isoform d, putative | | | |
| 12649.m00085 | Bm1_02155 | Delta5 fatty acid | 12649 | 147 | 1019 |
| | | desaturase-related | | | |
| 12653.m00011 | Bm1_02195 | hypothetical protein | 12653 | 1472 | 2549 |
| 12688.m00058 | Bm1_02410 | hypothetical protein | 12688 | 1685 | 2152 |
| 12694.m00015 | Bm1_02455 | WW domain containing | 12694 | 192 | 3186 |
| | | protein | | | |
| 12698.m00331 | Bm1_02520 | 1300013D05Rik protein- | 12698 | 35443 | 33408 |
| | | related | | | |
| 12701.m00057 | Bm1_02565 | 40S ribosomal protein | 12701 | 4689 | 4996 |
| | | S12.-related | | | |
| 12715.m00106 | Bm1_02630 | hypothetical protein | 12715 | 119 | 4508 |
| 12720.m00024 | Bm1_02675 | GYF domain containing | 12720 | 3836 | 2233 |
| | | protein | | | |
| 12778.m00055 | Bm1_03010 | PRO0477p-related | 12778 | 22131 | 22549 |
| 12791.m00115 | Bm1_03155 | gag protein-related | 12791 | 1786 | 3048 |
| 12823.m00025 | Bm1_03370 | LD15209p, putative | 12823 | 1206 | 436 |
| 12845.m00017 | Bm1_03495 | Kunitz/Bovine pancreatic | 12845 | 1710 | 725 |
| | | trypsin inhibitor domain | | | |
| | | containing protein | | | |
| 12870.m00009 | Bm1_03645 | Warthog protein-related | 12870 | 2461 | 392 |
| 12888.m00015 | Bm1_03765 | hypothetical protein | 12888 | 254 | 101 |
| 12888.m00016 | Bm1_03770 | FLYWCH zinc finger | 12888 | 1922 | 1186 |
| | | domain containing | | | |
| | | protein | | | |
| 12902.m00216 | Bm1_03840 | Hypothetical protein | 12902 | 10037 | 9119 |
| 12902.m00224 | Bm1_03880 | hypothetical protein | 12902 | 39508 | 33744 |
| 12902.m00232 | Bm1_03920 | Cytochrome c oxidase | 12902 | 68068 | 70046 |
| | | subunit IV family protein | | | |
| 12927.m00019 | Bm1_04070 | Salivary glue protein | 12927 | 2537 | 8 |
| | | Sgs-3 precursor.-related | | | |
| 13001.m00046 | Bm1_04435 | Hypothetical protein | 13001 | 86 | 220 |
| 13047.m00009 | Bm1_04665 | 2,3- | 13047 | 2834 | 1308 |
| | | bisphosphoglycerate- | | | |
| | | independent | | | |
| | | phosphoglycerate | | | |
| | | mutase, putative | | | |
| 13058.m00015 | Bm1_04725 | Helix-loop-helix DNA- | 13058 | 1607 | 2715 |
| | | binding domain | | | |
| | | containing protein | | | |
| 13066.m00231 | Bm1_04775 | hypothetical protein | 13066 | 15360 | 16129 |
| 13066.m00233 | Bm1_04785 | AN1-like Zinc finger | 13066 | 22342 | 20390 |
| | | family protein | | | |
| 13066.m00250 | Bm1_04865 | DNA polymerase epsilon | 13066 | 100521 | 101389 |
| | | p17 subunit, putative | | | |
| 13068.m00024 | Bm1_04880 | NADH-ubiquinone | 13068 | 6910 | 5979 |
| | | oxidoreductase AGGG | | | |
| | | subunit homolog, | | | |
| | | mitochondrialprecursor.- | | | |
| | | related | | | |
| 13123.m00029 | Bm1_05160 | Troponin T.-related | 13123 | 139 | 3283 |
| 13128.m00013 | Bm1_05190 | hypothetical protein | 13128 | 2015 | 268 |
| 13132.m00016 | Bm1_05210 | hypothetical protein | 13132 | 1502 | 1583 |
| 13143.m00017 | Bm1_05345 | WD-repeat protein 3.- | 13143 | 9537 | 8808 |
| | | related | | | |
| 13154.m00137 | Bm1_05435 | conserved hypothetical | 13154 | 25400 | 22653 |
| | | protein | | | |
| 13156.m00091 | Bm1_05470 | hypothetical protein | 13156 | 5006 | 7380 |
| 13192.m00017 | Bm1_05820 | hypothetical protein | 13192 | 2102 | 2272 |
| 13204.m00046 | Bm1_05895 | Gex interacting protein | 13204 | 10131 | 16129 |
| | | protein 16, isoform d- | | | |
| | | related | | | |
| 13210.m00168 | Bm1_05960 | Patched family protein | 13210 | 59598 | 53762 |
| 13223.m00106 | Bm1_06045 | conserved hypothetical | 13223 | 11422 | 10670 |
| | | protein | | | |
| 13236.m00034 | Bm1_06130 | Nuclear anchorage | 13236 | 13917 | 11658 |
| | | protein 1-related | | | |
| 13247.m00698 | Bm1_06290 | hypothetical protein | 13247 | 106697 | 105212 |
| 13247.m00702 | Bm1_06310 | protein R52.2-related | 13247 | 109582 | 109992 |
| 13247.m00708 | Bm1_06340 | hypothetical protein | 13247 | 140585 | 142998 |
| 13250.m00034 | Bm1_06460 | hypothetical protein | 13250 | 22829 | 21671 |
| 13260.m00102 | Bm1_06640 | Protein phosphatase | 13260 | 36634 | 38089 |
| | | inhibitor containing | | | |
| | | protein | | | |
| 13261.m00254 | Bm1_06655 | hypothetical protein | 13261 | 11032 | 8811 |
| 13261.m00256 | Bm1_06665 | Transmembrane amino | 13261 | 25615 | 29392 |
| | | acid transporter protein | | | |
| 13269.m00314 | Bm1_06785 | conserved hypothetical | 13269 | 41222 | 43942 |
| | | protein | | | |
| 13278.m00098 | Bm1_06925 | F26F3.2 protein-related | 13278 | 2424 | 271 |
| 13294.m00109 | Bm1_07190 | hypothetical protein | 13294 | 18202 | 16400 |
| 13315.m00131 | Bm1_07440 | hypothetical protein | 13315 | 10342 | 10163 |
| 13315.m00133 | Bm1_07450 | hypothetical protein | 13315 | 13543 | 15318 |
| 13315.m00140 | Bm1_07485 | hypothetical protein | 13315 | 32298 | 32753 |
| 13322.m00190 | Bm1_07615 | Peroxin-3 family protein | 13322 | 10487 | 7810 |
| 13322.m00194 | Bm1_07635 | GM16138p-related | 13322 | 26808 | 25963 |
| 13325.m00229 | Bm1_07680 | EB module family | 13325 | 19498 | 22956 |
| | | protein | | | |
| 13333.m00082 | Bm1_07780 | immunogenic protein 3, | 13333 | 13983 | 14581 |
| | | putative | | | |
| 13335.m00032 | Bm1_07795 | hypothetical protein | 13335 | 2418 | 3861 |
| 13350.m00131 | Bm1_07885 | SD09147p-related | 13350 | 11949 | 11314 |
| 13354.m00140 | Bm1_07925 | peroxisomal membrane | 13354 | 4698 | 3142 |
| | | anchor protein, putative | | | |
| 13356.m00233 | Bm1_08025 | F-box domain containing | 13356 | 11296 | 9151 |
| | | protein | | | |
| 13356.m00235 | Bm1_08035 | Hypothetical 36.0 kDa | 13356 | 20774 | 23113 |
| | | protein C45G9.5 in | | | |
| | | chromosome III.-related | | | |
| 13366.m00256 | Bm1_08175 | conserved hypothetical | 13366 | 13084 | 16928 |
| | | protein | | | |
| 13369.m00043 | Bm1_08225 | conserved hypothetical | 13369 | 10820 | 11384 |
| | | protein | | | |
| 13388.m00076 | Bm1_08450 | hypothetical protein | 13388 | 25185 | 26629 |
| 13398.m00096 | Bm1_08545 | Mediator protein 4- | 13398 | 33124 | 29280 |
| | | related | | | |
| 13400.m00320 | Bm1_08610 | Hypothetical protein | 13400 | 54710 | 51713 |
| 13409.m00048 | Bm1_08695 | trehalose-6-phosphate | 13409 | 6287 | 9952 |
| | | synthase-related | | | |
| 13411.m00122 | Bm1_08735 | hypothetical protein | 13411 | 12237 | 14686 |
| 13411.m00124 | Bm1_08745 | hypothetical protein | 13411 | 19434 | 17983 |
| 13415.m00462 | Bm1_08915 | hypothetical protein | 13415 | 164259 | 170446 |
| 13444.m00042 | Bm1_09120 | Hypothetical protein | 13444 | 3311 | 2059 |
| 13449.m00084 | Bm1_09160 | conserved hypothetical | 13449 | 9152 | 11804 |
| | | protein | | | |
| 13460.m00194 | Bm1_09225 | HIT zinc finger family | 13460 | 5999 | 3075 |
| | | protein | | | |
| 13464.m00238 | Bm1_09270 | Skp1 related (ubiquitin | 13464 | 8468 | 14561 |
| | | ligase complex | | | |
| | | component) protein 18- | | | |
| | | like | | | |
| 13465.m00049 | Bm1_09360 | conserved hypothetical | 13465 | 8139 | 1009 |
| | | protein | | | |
| 13473.m00077 | Bm1_09495 | conserved hypothetical | 13473 | 24111 | 20711 |
| | | protein | | | |
| 13491.m00026 | Bm1_09640 | Nematode cuticle | 13491 | 4247 | 1874 |
| | | collagen N-terminal | | | |
| | | domain containing | | | |
| | | protein | | | |
| 13497.m00179 | Bm1_09670 | NADH-ubiquinone | 13497 | 13634 | 16174 |
| | | oxidoreductase B22 | | | |
| | | subunit.-related | | | |
| 13527.m00034 | Bm1_09930 | kinesin light chain, | 13527 | 4104 | 4196 |
| | | putative | | | |
| 13534.m00021 | Bm1_09975 | hypothetical protein | 13534 | 1669 | 4914 |
| 13558.m00035 | Bm1_10140 | Zinc finger, C2H2 type | 13558 | 1787 | 10 |
| | | family protein | | | |
| 13562.m00095 | Bm1_10195 | Hypothetical protein | 13562 | 26053 | 26792 |
| 13572.m00009 | Bm1_10215 | Calcium-binding | 13572 | 1904 | 2909 |
| | | protein.-related | | | |
| 13579.m00037 | Bm1_10260 | gene model 83, putative | 13579 | 189 | 327 |
| 13588.m00011 | Bm1_10315 | Long protein 1, isoform | 13588 | 1070 | 1198 |
| | | b, putative | | | |
| 13604.m00012 | Bm1_10425 | hypothetical protein | 13604 | 1914 | 1989 |
| 13613.m00040 | Bm1_10475 | hypothetical protein | 13613 | 7719 | 4650 |
| 13632.m00183 | Bm1_10660 | Hypothetical 20.9 kDa | 13632 | 383 | 1043 |
| | | protein in PLB1-HXT2 | | | |
| | | intergenic region.- | | | |
| | | related | | | |
| 13644.m00292 | Bm1_10835 | hypothetical protein | 13644 | 82172 | 78526 |
| 13645.m00040 | Bm1_10860 | hypothetical protein | 13645 | 9747 | 13525 |
| 13667.m00039 | Bm1_11075 | RNA-dependent helicase, | 13667 | 19581 | 14799 |
| | | putative | | | |
| 13705.m00023 | Bm1_11340 | C2—HC type zinc finger | 13705 | 1872 | 2063 |
| | | protein C.e-MyT1, | | | |
| | | putative | | | |
| 13736.m00406 | Bm1_11590 | Hypothetical 30.1 kDa | 13736 | 49212 | 51909 |
| | | protein ZC434.4 in | | | |
| | | chromosome I.-related | | | |
| 13736.m00410 | Bm1_11565 | predicted protein | 13736 | | |
| 13781.m00021 | Bm1_11825 | hypothetical protein | 13781 | 3393 | 239 |
| 13785.m00207 | Bm1_11840 | hypothetical protein | 13785 | 9166 | 8088 |
| 13847.m00044 | Bm1_12400 | hypothetical protein | 13847 | 11222 | 12502 |
| 13890.m00008 | Bm1_12550 | hypothetical protein | 13890 | 564 | 1891 |
| 13920.m00451 | Bm1_12855 | hypothetical protein | 13920 | 125731 | 121489 |
| 13939.m00060 | Bm1_13005 | hypothetical protein | 13939 | 575 | 1045 |
| 13941.m00057 | Bm1_13030 | conserved hypothetical | 13941 | 3641 | 466 |
| | | protein | | | |
| 13944.m00013 | Bm1_13050 | Hypothetical protein | 13944 | 842 | 85 |
| 13955.m00009 | Bm1_13150 | Barrier-to- | 13955 | 2073 | 2198 |
| | | autointegration factor 1, | | | |
| | | putative | | | |
| 13961.m00024 | Bm1_13170 | conserved hypothetical | 13961 | 3393 | 541 |
| | | protein | | | |
| 13965.m00025 | Bm1_13195 | hypothetical protein | 13965 | 1875 | 112 |
| 14009.m00173 | Bm1_13520 | Hypothetical protein- | 14009 | 27451 | 29497 |
| | | conserved | | | |
| 14012.m00014 | Bm1_13550 | conserved hypothetical | 14012 | 196 | 2657 |
| | | protein | | | |
| 14015.m00090 | Bm1_13600 | major sperm protein 2, | 14015 | 21571 | 21054 |
| | | putative cytoskeletal | | | |
| | | MSP | | | |
| 14015.m00091 | Bm1_13605 | Major Sperm Protein | 14015 | 22020 | 24645 |
| | | (MSP), putative | | | |
| | | cytoskeletal MSP | | | |
| 14033.m00022 | Bm1_13715 | Fras1 protein-related | 14033 | 1642 | 294 |
| 14039.m00119 | Bm1_13915 | Nematode astacin | 14039 | 54118 | 56826 |
| | | protease protein 9, | | | |
| | | isoform c-related | | | |
| 14041.m00080 | Bm1_13965 | M-phase | 14041 | 43582 | 44602 |
| | | phosphoprotein-related | | | |
| 14046.m00194 | Bm1_14055 | ShTK domain containing | 14046 | 50144 | 49529 |
| | | protein | | | |
| 14052.m00191 | Bm1_14115 | hypothetical protein | 14052 | 39706 | 43773 |
| 14058.m00558 | Bm1_14240 | PWWP domain | 14058 | 23922 | 19492 |
| | | containing protein | | | |
| 14058.m00575 | Bm1_14325 | hypothetical protein | 14058 | 79555 | 78604 |
| 14058.m00576 | Bm1_14330 | Mitochondrial ATP | 14058 | 86211 | 87347 |
| | | synthase coupling factor | | | |
| | | 6 family protein | | | |
| 14058.m00579 | Bm1_14345 | Helix-loop-helix DNA- | 14058 | 101813 | 104473 |
| | | binding domain | | | |
| | | containing protein | | | |
| 14094.m00132 | Bm1_14650 | hypothetical protein | 14094 | 23688 | 16738 |
| 14094.m00138 | Bm1_14680 | PDZ domain containing | 14094 | 32604 | 34066 |
| | | protein | | | |
| 14097.m00079 | Bm1_14750 | Ubiquinol-cytochrome C | 14097 | 23000 | 25184 |
| | | reductase hinge protein | | | |
| 14122.m00164 | Bm1_14965 | hypothetical protein | 14122 | 23906 | 26065 |
| 14151.m00029 | Bm1_15165 | hypothetical protein | 14151 | 7109 | 7699 |
| 14164.m00121 | Bm1_15245 | RH17657p-related | 14164 | 14912 | 13414 |
| 14196.m00041 | Bm1_15510 | RE18450p, putative | 14196 | 1527 | 3260 |
| 14208.m00914 | Bm1_15680 | hypothetical protein | 14208 | 34974 | 36381 |
| 14219.m00026 | Bm1_15840 | Surfeit locus protein 6 | 14219 | 5244 | 4204 |
| | | containing protein | | | |
| 14229.m00038 | Bm1_15990 | RE06140p-related | 14229 | 6203 | 3847 |
| 14230.m00222 | Bm1_16040 | hypothetical protein | 14230 | 34438 | 33660 |
| 14237.m00398 | Bm1_16245 | symbol-related | 14237 | 60928 | 63143 |
| 14239.m00342 | Bm1_16340 | hypothetical protein | 14239 | 35560 | 39598 |
| 14248.m00663 | Bm1_16530 | Hint module family | 14248 | 1711 | 7852 |
| | | protein | | | |
| 14248.m00664 | Bm1_16540 | hypothetical protein | 14248 | 13460 | 14984 |
| 14248.m00667 | Bm1_16555 | hypothetical protein | 14248 | 26651 | 25215 |
| 14250.m00292 | Bm1_16675 | hypothetical protein | 14250 | 22580 | 25202 |
| 14250.m00295 | Bm1_16685 | hypothetical protein | 14250 | 34216 | 28377 |
| 14250.m00299 | Bm1_16705 | conserved hypothetical | 14250 | 42808 | 41455 |
| | | protein | | | |
| 14253.m00158 | Bm1_16780 | hypothetical protein | 14253 | 94209 | 96292 |
| 14276.m00246 | Bm1_17070 | Leucine Rich Repeat | 14276 | 23570 | 27964 |
| | | family protein | | | |
| 14279.m00042 | Bm1_17120 | Hyaluronan/mRNA | 14279 | 2038 | 4764 |
| | | binding family protein | | | |
| 14282.m00452 | Bm1_17210 | conserved hypothetical | 14282 | 71357 | 75320 |
| | | protein | | | |
| 14284.m00386 | Bm1_17305 | Hypothetical protein | 14284 | 83699 | 81725 |
| 14318.m00072 | Bm1_17810 | vacuolar ATP synthase | 14318 | 13564 | 12905 |
| | | subunit H, putative | | | |
| 14328.m00023 | Bm1_17930 | chitin synthase 2 (chs-2) | 14328 | 4006 | 83 |
| | | fragment | | | |
| 14341.m00010 | Bm1_18060 | hypothetical protein | 14341 | 1222 | 3936 |
| 14348.m00100 | Bm1_18115 | hypothetical protein | 14348 | 7241 | 14740 |
| 14355.m00214 | Bm1_18195 | Serine/threonine protein | 14355 | 14483 | 15779 |
| | | phosphatase PP1 | | | |
| | | isozyme 1, putative | | | |
| 14379.m00149 | Bm1_18685 | conserved hypothetical | 14379 | 16552 | 18189 |
| | | protein | | | |
| 14379.m00151 | Bm1_18695 | Nematode cuticle | 14379 | 29010 | 30713 |
| | | collagen N-terminal | | | |
| | | domain containing | | | |
| | | protein | | | |
| 14386.m00052 | Bm1_18760 | conserved hypothetical | 14386 | 3732 | 2621 |
| | | protein | | | |
| 14387.m00349 | Bm1_18845 | GRIM-19 protein | 14387 | 83445 | 85619 |
| 14396.m00009 | Bm1_19065 | conserved hypothetical | 14396 | 1522 | 6613 |
| | | protein | | | |
| 14409.m00256 | Bm1_19285 | Innexin family protein | 14409 | 109344 | 103608 |
| 14411.m00015 | Bm1_19290 | hypothetical protein | 14411 | 765 | 201 |
| 14417.m00065 | Bm1_19380 | hypothetical protein | 14417 | 16685 | 19318 |
| 14418.m00019 | Bm1_19390 | Hypothetical protein | 14418 | 1177 | 2236 |
| 14420.m00010 | Bm1_19420 | hypothetical protein | 14420 | 168 | 974 |
| 14421.m00015 | Bm1_19425 | MNN4 protein.-related | 14421 | 3261 | 2343 |
| 14423.m00101 | Bm1_19440 | hypothetical protein | 14423 | 19051 | 23364 |
| 14450.m00173 | Bm1_19655 | Hypothetical protein | 14450 | 24648 | 22911 |
| 14479.m00132 | Bm1_19985 | cuticle collagen 2 | 14479 | 10715 | 13407 |
| | | precursor, putative | | | |
| 14489.m00060 | Bm1_20120 | PDZ domain containing | 14489 | 20528 | 19152 |
| | | protein | | | |
| 14522.m00057 | Bm1_20495 | hypothetical protein | 14522 | 14271 | 16331 |
| 14535.m00021 | Bm1_20745 | adenosine deaminase | 14535 | 2950 | 1588 |
| | | ADR-1C, putative | | | |
| 14538.m00475 | Bm1_20785 | hypothetical protein | 14538 | 36359 | 29660 |
| 14539.m00055 | Bm1_20840 | calcium-binding protein, | 14539 | 5451 | 4903 |
| | | putative | | | |
| 14554.m00230 | Bm1_21040 | Hypothetical thiol | 14554 | 21383 | 19767 |
| | | protease C06G4.2 in | | | |
| | | chromosome III.-related | | | |
| 14569.m00218 | Bm1_21225 | hypothetical protein | 14569 | 30318 | 28980 |
| 14569.m00224 | Bm1_21255 | Chitin binding | 14569 | 81686 | 68369 |
| | | Peritrophin-A domain | | | |
| | | containing protein | | | |
| 14588.m00024 | Bm1_21530 | hypothetical protein | 14588 | 14493 | 10297 |
| 14590.m00346 | Bm1_21620 | Profilin family protein | 14590 | 48140 | 49570 |
| 14592.m00176 | Bm1_21655 | hypothetical protein | 14592 | 35852 | 34826 |
| 14593.m00155 | Bm1_21695 | hypothetical protein | 14593 | 33683 | 38688 |
| 14599.m00264 | Bm1_21815 | hypothetical protein | 14599 | 58082 | 56385 |
| 14599.m00266 | Bm1_21825 | hypothetical protein | 14599 | 63069 | 61135 |
| 14601.m00160 | Bm1_21885 | SWIB/MDM2 domain | 14601 | 11785 | 7195 |
| | | containing protein | | | |
| 14603.m00270 | Bm1_21970 | conserved hypothetical | 14603 | 46845 | 50470 |
| | | protein | | | |
| 14628.m00170 | Bm1_22440 | Homeobox protein | 14628 | 3335 | 520 |
| | | goosecoid, putative | | | |
| 14631.m00037 | Bm1_22500 | hypothetical protein | 14631 | 9722 | 14473 |
| 14632.m00150 | Bm1_22525 | DNA-(Apurinic or | 14632 | 41365 | 37539 |
| | | apyrimidinic site) lyase- | | | |
| | | related | | | |
| 14634.m00536 | Bm1_22560 | hypothetical protein | 14634 | 19692 | 19799 |
| 14637.m00177 | Bm1_22670 | conserved hypothetical | 14637 | 21317 | 23555 |
| | | protein | | | |
| 14638.m00118 | Bm1_22725 | RNA dependent RNA | 14638 | 20712 | 35208 |
| | | polymerase family | | | |
| | | protein | | | |
| 14640.m00210 | Bm1_22765 | hypothetical protein | 14640 | 29571 | 27602 |
| 14643.m00073 | Bm1_22805 | Hypothetical protein | 14643 | 8865 | 8466 |
| 14643.m00076 | Bm1_22820 | hypothetical protein | 14643 | 33914 | 33180 |
| 14649.m00093 | Bm1_22905 | hypothetical protein | 14649 | 9460 | 5878 |
| 14652.m00402 | Bm1_22990 | actin-depolymerizing | 14652 | 13756 | 12623 |
| | | factor 1, putative | | | |
| 14652.m00406 | Bm1_23010 | hypothetical protein | 14652 | 29119 | 27550 |
| 14652.m00407 | Bm1_23015 | hypothetical protein, | 14652 | 32666 | 30721 |
| | | conserved | | | |
| 14653.m00286 | Bm1_23080 | ATP synthase f chain, | 14653 | 41458 | 40460 |
| | | mitochondrial.-related | | | |
| 14656.m00217 | Bm1_23135 | Hypothetical protein | 14656 | 516 | 1535 |
| 14656.m00226 | Bm1_23180 | ribosomal protein L32 | 14656 | 54103 | 55318 |
| | | containing protein | | | |
| 14668.m00161 | Bm1_23370 | Ulp1 protease family, C- | 14668 | 24911 | 27782 |
| | | terminal catalytic | | | |
| | | domain containing | | | |
| | | protein | | | |
| 14669.m00054 | Bm1_23380 | Zinc finger, C2H2 type | 14669 | 9736 | 12495 |
| | | family protein | | | |
| 14669.m00055 | Bm1_23385 | conserved hypothetical | 14669 | 15485 | 19178 |
| | | protein | | | |
| 14677.m00168 | Bm1_23555 | UcrQ family protein | 14677 | 15775 | 16684 |
| 14677.m00171 | Bm1_23570 | conserved hypothetical | 14677 | 33001 | 29833 |
| | | protein | | | |
| 14683.m00062 | Bm1_23670 | FRG1 protein homolog, | 14683 | 17216 | 17368 |
| | | putative | | | |
| 14696.m00216 | Bm1_23935 | heavy metal-associated | 14696 | 30783 | 31456 |
| | | domain containing | | | |
| | | protein | | | |
| 14704.m00455 | Bm1_24165 | TolA protein.-related | 14704 | 49404 | 47337 |
| 14715.m01243 | Bm1_24555 | hypothetical protein | 14715 | 60146 | 61013 |
| 14715.m01245 | Bm1_24565 | conserved hypothetical | 14715 | 72788 | 69063 |
| | | protein | | | |
| 14715.m01248 | Bm1_24580 | ATP synthase e chain, | 14715 | 85772 | 86557 |
| | | mitochondrial.-related | | | |
| 14715.m01255 | Bm1_24615 | ATP synthase B chain, | 14715 | 140193 | 137886 |
| | | mitochondrial precursor, | | | |
| | | putative | | | |
| 14735.m00112 | Bm1_25025 | Spc97/Spc98 family | 14735 | 14161 | 21918 |
| | | protein | | | |
| 14740.m00011 | Bm1_25060 | Nematode cuticle | 14740 | 3111 | 1193 |
| | | collagen N-terminal | | | |
| | | domain containing | | | |
| | | protein | | | |
| 14746.m00118 | Bm1_25120 | Tudor domain containing | 14746 | 13616 | 16441 |
| | | protein | | | |
| 14758.m00155 | Bm1_25285 | Prion-like--related | 14758 | 3779 | 5015 |
| 14764.m00052 | Bm1_25440 | hypothetical protein | 14764 | 1010 | 882 |
| 14770.m00165 | Bm1_25640 | hypothetical protein | 14770 | 10440 | 16450 |
| 14773.m00912 | Bm1_25750 | Lipase family protein | 14773 | 88323 | 85780 |
| 14773.m00925 | Bm1_25810 | Hepatocellular | 14773 | 181787 | 182284 |
| | | carcinoma-associated | | | |
| | | antigen 127, putative | | | |
| 14776.m00033 | Bm1_25910 | RIKEN cDNA | 14776 | 9279 | 7798 |
| | | 2610002M06, putative | | | |
| 14786.m00011 | Bm1_26170 | hypothetical protein | 14786 | 773 | 2150 |
| 14799.m00204 | Bm1_26345 | Hypothetical protein | 14799 | 7761 | 4406 |
| 14820.m00017 | Bm1_26495 | hypothetical protein | 14820 | 366 | 516 |
| 14832.m00025 | Bm1_26605 | UPF0279 protein | 14832 | 4821 | 5981 |
| | | C14orf129 homolog.- | | | |
| | | related | | | |
| 14853.m00060 | Bm1_26745 | hypothetical protein | 14853 | 2857 | 315 |
| 14878.m00010 | Bm1_27030 | Talin 1, putative | 14878 | 280 | 701 |
| 14900.m00208 | Bm1_27220 | hypothetical protein | 14900 | 36652 | 30195 |
| 14902.m00008 | Bm1_27240 | hypothetical protein | 14902 | 182 | 268 |
| 14905.m00133 | Bm1_27280 | hypothetical protein | 14905 | 48057 | 53498 |
| 14907.m00564 | Bm1_27330 | conserved hypothetical | 14907 | 53267 | 52044 |
| | | protein | | | |
| 14912.m00013 | Bm1_27455 | Lectin C-type domain | 14912 | 2592 | 1279 |
| | | containing protein | | | |
| 14916.m00477 | Bm1_27515 | hypothetical protein | 14916 | 47168 | 46186 |
| 14917.m00318 | Bm1_27615 | bZIP transcription factor | 14917 | 8334 | 6572 |
| | | family protein | | | |
| 14917.m00336 | Bm1_27705 | 7B2-related | 14917 | 140405 | 137941 |
| 14921.m00195 | Bm1_28035 | Hypothetical protein | 14921 | 3321 | 1512 |
| 14921.m00200 | Bm1_28060 | Hypothetical protein | 14921 | 49046 | 48087 |
| 14924.m00113 | Bm1_28165 | hypothetical protein | 14924 | 50496 | 51915 |
| 14929.m00388 | Bm1_28315 | conserved hypothetical | 14929 | 51946 | 42468 |
| | | protein | | | |
| 14930.m00348 | Bm1_28490 | conserved hypothetical | 14930 | 141151 | 144901 |
| | | protein | | | |
| 14932.m00515 | Bm1_28625 | Mitochondrial | 14932 | 81154 | 79346 |
| | | glycoprotein | | | |
| 14932.m00524 | Bm1_28670 | hypothetical protein | 14932 | 123272 | 124217 |
| 14937.m00488 | Bm1_28945 | hypothetical protein | 14937 | 70256 | 73739 |
| 14938.m00331 | Bm1_29000 | hypothetical protein | 14938 | 13950 | 12625 |
| 14940.m00174 | Bm1_29140 | hypothetical protein | 14940 | 14677 | 15750 |
| 14944.m00531 | Bm1_29320 | hypothetical protein | 14944 | 3910 | 2900 |
| 14944.m00552 | Bm1_29430 | Cytochrome c oxidase | 14944 | 114518 | 115864 |
| | | polypeptide Vb, | | | |
| | | mitochondrial | | | |
| | | precursor.-related | | | |
| 14944.m00553 | Bm1_29435 | Hypothetical protein | 14944 | 119653 | 118456 |
| 14946.m00545 | Bm1_29610 | hypothetical protein | 14946 | 116312 | 113520 |
| 14947.m01145 | Bm1_29715 | Hypothetical protein | 14947 | 231798 | 233231 |
| 14950.m01792 | Bm1_29880 | Ubiquitin carboxyl- | 14950 | 30741 | 34678 |
| | | terminal hydrolase | | | |
| | | family protein | | | |
| 14950.m01808 | Bm1_29960 | von Willebrand factor | 14950 | 115733 | 107271 |
| | | type A domain | | | |
| | | containing protein | | | |
| 14950.m01833 | Bm1_30085 | Apoptosis regulator | 14950 | 226346 | 221668 |
| | | proteins, Bcl-2 family | | | |
| | | protein | | | |
| 14950.m01862 | Bm1_30230 | Hypothetical 19.4 kDa | 14950 | 399978 | 401134 |
| | | protein ZC395.10 in | | | |
| | | chromosome III.-related | | | |
| 14953.m00217 | Bm1_30505 | Neurotransmitter-gated | 14953 | 31862 | 28779 |
| | | ion-channel | | | |
| | | transmembrane region | | | |
| | | family protein | | | |
| 14954.m01603 | Bm1_30695 | hypothetical protein | 14954 | 228908 | 230217 |
| 14954.m01678 | Bm1_31055 | RNA recognition motif | 14954 | 677472 | 683353 |
| | | containing protein, | | | |
| | | putative | | | |
| 14954.m01709 | Bm1_31210 | Zinc finger, C2H2 type | 14954 | 873180 | 876894 |
| | | family protein | | | |
| 14956.m00513 | Bm1_31660 | hypothetical protein | 14956 | 239559 | 237010 |
| 14958.m00350 | Bm1_31870 | Surfeit locus protein 5 | 14958 | 99724 | 101317 |
| | | containing protein | | | |
| 14961.m04897 | Bm1_32025 | hypothetical protein | 14961 | 30034 | 29813 |
| 14961.m04921 | Bm1_32145 | Cuticle collagen dpy-7 | 14961 | 189438 | 188307 |
| | | precursor, putative | | | |
| 14961.m04928 | Bm1_32180 | hypothetical protein | 14961 | 261282 | 259422 |
| 14961.m04944 | Bm1_32260 | conserved hypothetical | 14961 | 346907 | 342655 |
| | | protein | | | |
| 14961.m04948 | Bm1_32280 | hypothetical protein | 14961 | 385468 | 387037 |
| 14961.m05035 | Bm1_32720 | hypothetical protein | 14961 | 909381 | 906543 |
| 14961.m05037 | Bm1_32730 | LBP/BPI/CETP family, | 14961 | 948962 | 953135 |
| | | C-terminal domain | | | |
| | | containing protein | | | |
| 14961.m05066 | Bm1_32875 | Calponin homolog | 14961 | 1135670 | 1132938 |
| | | OV9M.-related | | | |
| 14961.m05089 | Bm1_32990 | Apical junction molecule | 14961 | 1285908 | 1277047 |
| | | protein 1, isoform d- | | | |
| | | related | | | |
| 14961.m05095 | Bm1_33020 | LAMP family protein Imp- | 14961 | 1323327 | 1324602 |
| | | 1 precursor.-related | | | |
| 14961.m05104 | Bm1_33065 | hypothetical protein | 14961 | 1364571 | 1367984 |
| 14961.m05112 | Bm1_33105 | hypothetical protein | 14961 | 1441631 | 1438388 |
| 14961.m05133 | Bm1_33205 | Protein cab-1.-related | 14961 | 1553352 | 1550253 |
| 14961.m05175 | Bm1_33410 | CG32584-PB-related | 14961 | 1776707 | 1777493 |
| 14961.m05181 | Bm1_33440 | Innexin family protein | 14961 | 1822356 | 1826065 |
| 14961.m05194 | Bm1_33500 | hypothetical protein | 14961 | 1892771 | 1890512 |
| 14961.m05207 | Bm1_33565 | conserved hypothetical | 14961 | 1977516 | 1974679 |
| | | protein | | | |
| 14961.m05209 | Bm1_33575 | hypothetical protein | 14961 | 1982685 | 1980209 |
| 14961.m05223 | Bm1_33635 | hypothetical protein | 14961 | 2078206 | 2080034 |
| 14961.m05249 | Bm1_33765 | hypothetical protein | 14961 | 2224566 | 2221646 |
| 14961.m05250 | Bm1_33770 | zgc: 92910-related | 14961 | 2224854 | 2226035 |
| 14961.m05267 | Bm1_33855 | Ubiquinol-cytochrome C | 14961 | 2352628 | 2355445 |
| | | chaperone family protein | | | |
| 14961.m05319 | Bm1_34110 | hypothetical protein | 14961 | 2678053 | 2679408 |
| 14961.m05325 | Bm1_34145 | RE35789p-related | 14961 | 2731520 | 2730227 |
| 14961.m05347 | Bm1_34260 | M-phase | 14961 | 2912892 | 2916031 |
| | | phosphoprotein, mpp8, | | | |
| | | putative | | | |
| 14961.m05378 | Bm1_33865 | predicted protein | 14961 | | |
| 14962.m00670 | Bm1_34425 | Ctr copper transporter | 14962 | 112000 | 113747 |
| | | family protein | | | |
| 14962.m00674 | Bm1_34445 | zgc: 91831-related | 14962 | 127919 | 126898 |
| 14963.m01764 | Bm1_34455 | amine oxidase, flavin- | 14963 | 4676 | 1119 |
| | | containing-related | | | |
| 14963.m01784 | Bm1_34560 | CGI-115 protein-related | 14963 | 176010 | 177363 |
| 14967.m01533 | Bm1_35045 | hypothetical protein | 14967 | 174476 | 175299 |
| 14967.m01536 | Bm1_35060 | Troponin family protein | 14967 | 184432 | 182488 |
| 14967.m01540 | Bm1_35075 | Innexin inx-3, putative | 14967 | 199141 | 202169 |
| 14967.m01549 | Bm1_35120 | PAN domain containing | 14967 | 271650 | 266430 |
| | | protein | | | |
| 14967.m01570 | Bm1_35215 | chitin synthase 1, chs-1 | 14967 | 413010 | 405294 |
| 14968.m01468 | Bm1_35390 | Slbp protein, putative | 14968 | 123987 | 126605 |
| 14968.m01469 | Bm1_35395 | Acyltransferase family | 14968 | 132176 | 136172 |
| | | protein | | | |
| 14968.m01473 | Bm1_35415 | 50S ribosomal protein | 14968 | 144841 | 145805 |
| | | L20.-related | | | |
| 14968.m01521 | Bm1_35660 | Succinate | 14968 | 398078 | 399458 |
| | | dehydrogenase, putative | | | |
| 14971.m02855 | Bm1_36075 | D10Ertd718e protein- | 14971 | 437939 | 437284 |
| | | related | | | |
| 14971.m02856 | Bm1_36080 | hypothetical protein | 14971 | 438706 | 444505 |
| 14971.m02876 | Bm1_36170 | PAN domain containing | 14971 | 515838 | 522920 |
| | | protein | | | |
| 14971.m02895 | Bm1_36265 | conserved hypothetical | 14971 | 634824 | 630514 |
| | | protein | | | |
| 14971.m02896 | Bm1_36270 | TspO/MBR family protein | 14971 | 637373 | 635976 |
| 14972.m06948 | Bm1_36295 | Resistance to inhibitors | 14972 | 20118 | 21489 |
| | | of cholinesterase protein | | | |
| | | 3-related | | | |
| 14972.m06952 | Bm1_36315 | spliced leader 175 kDa | 14972 | 49119 | 53484 |
| | | protein, putative | | | |
| 14972.m06956 | Bm1_36335 | conserved hypothetical | 14972 | 76833 | 77433 |
| | | protein | | | |
| 14972.m06981 | Bm1_36460 | hypothetical protein | 14972 | 223773 | 222536 |
| 14972.m07000 | Bm1_36555 | collagen col-34 - | 14972 | 333451 | 335360 |
| | | Caenorhabditis elegans, | | | |
| | | putative | | | |
| 14972.m07004 | Bm1_36575 | conserved hypothetical | 14972 | 402775 | 401379 |
| | | protein | | | |
| 14972.m07044 | Bm1_36765 | SD01790p-related | 14972 | 603450 | 612086 |
| 14972.m07139 | Bm1_37230 | conserved hypothetical | 14972 | 1255554 | 1253200 |
| | | protein | | | |
| 14972.m07141 | Bm1_37240 | ephrin EFN-4, putative | 14972 | 1279191 | 1280580 |
| 14972.m07143 | Bm1_37250 | RNA recognition motif. | 14972 | 1283517 | 1282842 |
| 14972.m07157 | Bm1_37315 | hypothetical protein | 14972 | 1339386 | 1338202 |
| 14972.m07193 | Bm1_37495 | conserved hypothetical | 14972 | 1566705 | 1571640 |
| | | protein | | | |
| 14972.m07197 | Bm1_37515 | hypothetical protein | 14972 | 1610896 | 1610169 |
| 14972.m07200 | Bm1_37530 | conserved hypothetical | 14972 | 1627567 | 1630330 |
| | | protein | | | |
| 14972.m07218 | Bm1_37610 | Destabilase family | 14972 | 1792336 | 1790947 |
| | | protein | | | |
| 14972.m07236 | Bm1_37705 | NHR1 homology to TAF | 14972 | 1955292 | 1958341 |
| | | family protein | | | |
| 14972.m07247 | Bm1_37760 | Zinc finger, C2H2 type | 14972 | 2035864 | 2042000 |
| | | family protein | | | |
| 14972.m07257 | Bm1_37810 | GGL domain containing | 14972 | 2092175 | 2093178 |
| | | protein | | | |
| 14972.m07267 | Bm1_37860 | NADH-dependent xylose | 14972 | 2167962 | 2170201 |
| | | reductase.-related | | | |
| 14972.m07286 | Bm1_37955 | hypothetical protein | 14972 | 2324626 | 2321009 |
| 14972.m07310 | Bm1_38065 | Clc-like | 14972 | 2493831 | 2491585 |
| 14972.m07318 | Bm1_38105 | hypothetical protein | 14972 | 2554836 | 2551961 |
| 14972.m07319 | Bm1_38110 | hypothetical protein | 14972 | 2559707 | 2556648 |
| 14972.m07321 | Bm1_38120 | hypothetical protein | 14972 | 2568777 | 2565415 |
| 14972.m07329 | Bm1_38160 | Fatty acid desaturase | 14972 | 2611543 | 2607670 |
| | | family protein | | | |
| 14972.m07378 | Bm1_38400 | Conserved hypothetical | 14972 | 2962953 | 2963607 |
| | | protein, putative | | | |
| 14972.m07383 | Bm1_38425 | 3-5 exonuclease family | 14972 | 3004127 | 3000790 |
| | | protein | | | |
| 14972.m07385 | Bm1_38435 | Conserved hypothetical | 14972 | 3024906 | 3025501 |
| | | protein, putative | | | |
| 14972.m07421 | Bm1_38610 | conserved hypothetical | 14972 | 3348979 | 3342705 |
| | | protein | | | |
| 14972.m07477 | Bm1_38875 | hypothetical protein | 14972 | 3736674 | 3735991 |
| 14972.m07478 | Bm1_38880 | Mitochondrial ATP | 14972 | 3737435 | 3738433 |
| | | synthase g subunit | | | |
| | | family protein | | | |
| 14972.m07542 | Bm1_39200 | hypothetical protein | 14972 | 4211804 | 4208933 |
| 14972.m07555 | Bm1_39265 | GH05862p-related | 14972 | 4273123 | 4274728 |
| 14972.m07565 | Bm1_39315 | Zinc finger, C2H2 type | 14972 | 4322507 | 4321891 |
| | | family protein | | | |
| 14972.m07569 | Bm1_39335 | conserved hypothetical | 14972 | 4335987 | 4339481 |
| | | protein | | | |
| 14972.m07582 | Bm1_39400 | BED zinc finger family | 14972 | 4424210 | 4426962 |
| | | protein | | | |
| 14972.m07626 | Bm1_39610 | hypothetical protein | 14972 | 4723625 | 4716074 |
| 14972.m07663 | Bm1_39790 | conserved hypothetical | 14972 | 4979146 | 4974663 |
| | | protein | | | |
| 14972.m07776 | Bm1_40345 | conserved hypothetical | 14972 | 5729018 | 5730606 |
| | | protein | | | |
| 14972.m07819 | Bm1_40540 | conserved hypothetical | 14972 | 5989038 | 5992470 |
| | | protein | | | |
| 14972.m07877 | Bm1_40800 | Helix-loop-helix DNA- | 14972 | 6373334 | 6371882 |
| | | binding domain | | | |
| | | containing protein | | | |
| 14972.m07922 | Bm1_37570 | predicted protein | 14972 | 1728585 | 1721938 |
| 14972.m07927 | Bm1_38270 | predicted protein | 14972 | 2775572 | 2771252 |
| 14972.m07928 | Bm1_38370 | predicted protein | 14972 | 2925519 | 2926537 |
| 14972.m07934 | Bm1_39000 | predicted protein | 14972 | 3937484 | 3934948 |
| 14973.m02594 | Bm1_40975 | hypothetical protein | 14973 | 105395 | 103306 |
| 14973.m02604 | Bm1_41030 | conserved hypothetical | 14973 | 156180 | 158084 |
| | | protein | | | |
| 14973.m02617 | Bm1_41110 | conserved hypothetical | 14973 | 233397 | 231475 |
| | | protein | | | |
| 14973.m02628 | Bm1_41180 | Conserved hypothetical | 14973 | 297573 | 296744 |
| | | protein, putative | | | |
| 14973.m02637 | Bm1_41220 | Helix-loop-helix DNA- | 14973 | 347444 | 348724 |
| | | binding domain | | | |
| | | containing protein | | | |
| 14973.m02692 | Bm1_41495 | Gex interacting protein | 14973 | 724480 | 728710 |
| | | protein 4, isoform c- | | | |
| | | related | | | |
| 14973.m02699 | Bm1_41530 | BED zinc finger family | 14973 | 785472 | 788815 |
| | | protein | | | |
| 14973.m02709 | Bm1_41565 | hAT family dimerisation | 14973 | 874608 | 881987 |
| | | domain containing | | | |
| | | protein | | | |
| 14973.m02715 | Bm1_41590 | Mitochondrial import | 14973 | 935297 | 933887 |
| | | inner membrane | | | |
| | | translocase subunit | | | |
| | | Tim17 family protein | | | |
| 14973.m02724 | Bm1_41635 | hypothetical protein | 14973 | 979881 | 974196 |
| 14974.m00805 | Bm1_41700 | Zn-finger in Ran binding | 14974 | 110944 | 114736 |
| | | protein and others | | | |
| | | containing protein | | | |
| 14974.m00820 | Bm1_41775 | UBA/TS-N domain | 14974 | 208164 | 212843 |
| | | containing protein | | | |
| 14974.m00848 | Bm1_41915 | NADH-ubiquinone | 14974 | 401842 | 402341 |
| | | oxidoreductase 18 kDa | | | |
| | | subunit, mitochondrial | | | |
| | | precursor.-related | | | |
| 14975.m04329 | Bm1_41995 | Conserved hypothetical | 14975 | 82747 | 80128 |
| | | protein, putative | | | |
| 14975.m04365 | Bm1_42145 | OTU-like cysteine | 14975 | 258873 | 256508 |
| | | protease family protein | | | |
| 14975.m04411 | Bm1_42370 | hypothetical protein | 14975 | 593588 | 585331 |
| 14975.m04436 | Bm1_42470 | hypothetical protein | 14975 | 742705 | 740384 |
| 14975.m04449 | Bm1_42535 | Tudor domain containing | 14975 | 849661 | 846729 |
| | | protein | | | |
| 14975.m04466 | Bm1_42620 | DNA segment, Chr 7, | 14975 | 904749 | 905826 |
| | | Wayne State University | | | |
| | | 180, expressed, putative | | | |
| 14975.m04471 | Bm1_42645 | hypothetical protein | 14975 | 914532 | 913197 |
| 14975.m04482 | Bm1_42700 | hypothetical protein | 14975 | 956252 | 954973 |
| 14975.m04488 | Bm1_42730 | conserved hypothetical | 14975 | 983441 | 982086 |
| | | protein | | | |
| 14975.m04537 | Bm1_42980 | Immunoglobulin I-set | 14975 | 1294164 | 1293295 |
| | | domain containing | | | |
| | | protein | | | |
| 14975.m04550 | Bm1_43045 | Dumpy: shorter than | 14975 | 1371133 | 1370569 |
| | | wild-type protein 10, | | | |
| | | isoform b, putative | | | |
| 14977.m04857 | Bm1_43075 | hypothetical protein | 14977 | 12164 | 7775 |
| 14977.m04900 | Bm1_43275 | membrane-associated | 14977 | 334342 | 331757 |
| | | RING-CH protein III, | | | |
| | | putative | | | |
| 14977.m04938 | Bm1_43465 | Temporarily assigned | 14977 | 552656 | 559748 |
| | | gene name protein 40, | | | |
| | | putative | | | |
| 14977.m04949 | Bm1_43515 | Hypothetical 36.5 kDa | 14977 | 618331 | 621448 |
| | | protein C56G2.3 in | | | |
| | | chromosome III.-related | | | |
| 14977.m04961 | Bm1_43570 | hypothetical protein | 14977 | 718696 | 726034 |
| 14977.m04992 | Bm1_43720 | conserved hypothetical | 14977 | 934807 | 933638 |
| | | protein | | | |
| 14977.m05040 | Bm1_43955 | hypothetical protein | 14977 | 1344816 | 1356262 |
| 14977.m05049 | Bm1_44000 | NADH-ubiquinone | 14977 | 1429728 | 1431068 |
| | | oxidoreductase ASHI | | | |
| | | subunit, mitochondrial | | | |
| | | precursor, putative | | | |
| 14977.m05056 | Bm1_44035 | hypothetical protein | 14977 | 1496735 | 1493619 |
| 14977.m05063 | Bm1_44070 | Zinc finger, C2H2 type | 14977 | 1601674 | 1604813 |
| | | family protein | | | |
| 14977.m05068 | Bm1_44095 | Conserved hypothetical | 14977 | 1623956 | 1625650 |
| | | protein, putative | | | |
| 14977.m05093 | Bm1_44220 | conserved hypothetical | 14977 | 1805138 | 1807992 |
| | | protein | | | |
| 14977.m05109 | Bm1_44295 | Helix-loop-helix DNA- | 14977 | 1938101 | 1943053 |
| | | binding domain | | | |
| | | containing protein | | | |
| 14979.m04465 | Bm1_44775 | hypothetical protein | 14979 | 505994 | 509063 |
| 14979.m04486 | Bm1_44890 | hypothetical protein | 14979 | 623304 | 630412 |
| 14979.m04520 | Bm1_45055 | Chitin binding | 14979 | 810826 | 812386 |
| | | Peritrophin-A domain | | | |
| | | containing protein | | | |
| 14979.m04521 | Bm1_45060 | hypothetical protein | 14979 | 814125 | 819352 |
| 14979.m04536 | Bm1_45135 | Conserved hypothetical | 14979 | 897478 | 895486 |
| | | protein, putative | | | |
| 14979.m04544 | Bm1_45175 | conserved hypothetical | 14979 | 985359 | 986554 |
| | | protein | | | |
| 14979.m04546 | Bm1_45185 | putative RNA binding | 14979 | 998637 | 996950 |
| | | protein | | | |
| 14979.m04553 | Bm1_45220 | CG7038-PA-related | 14979 | 1052050 | 1054378 |
| 14979.m04566 | Bm1_45285 | CG13018-PA, putative | 14979 | 1155460 | 1154666 |
| 14979.m04593 | Bm1_45405 | LD03534p-related | 14979 | 1312336 | 1313217 |
| 14979.m04631 | Bm1_45560 | hypothetical protein | 14979 | 1586951 | 1581993 |
| 14979.m04654 | Bm1_45665 | Mediator protein 11- | 14979 | 1731820 | 1731031 |
| | | related | | | |
| 14979.m04655 | Bm1_45670 | WH2 motif family protein | 14979 | 1733467 | 1737305 |
| 14980.m02723 | Bm1_46015 | F-box domain containing | 14980 | 321906 | 320502 |
| | | protein | | | |
| 14980.m02730 | Bm1_46050 | Zinc finger, C2H2 type | 14980 | 365468 | 368689 |
| | | family protein | | | |
| 14980.m02739 | Bm1_46095 | hypothetical protein | 14980 | 416147 | 415708 |
| 14980.m02747 | Bm1_46130 | hypothetical protein | 14980 | 440628 | 441172 |
| 14980.m02753 | Bm1_46160 | Conserved hypothetical | 14980 | 467374 | 468316 |
| | | protein, putative | | | |
| 14980.m02754 | Bm1_46165 | Chain A, Structure Of A | 14980 | 469157 | 468525 |
| | | Brca2-Dss1 Complex., | | | |
| | | putative | | | |
| 14980.m02796 | Bm1_46355 | hypothetical protein | 14980 | 736892 | 744026 |
| 14980.m02801 | Bm1_46380 | Probable dolichol- | 14980 | 771892 | 772179 |
| | | phosphate | | | |
| | | mannosyltransferase | | | |
| | | subunit 3, putative | | | |
| 14980.m02805 | Bm1_46400 | Nematode cuticle | 14980 | 796837 | 794518 |
| | | collagen N-terminal | | | |
| | | domain containing | | | |
| | | protein | | | |
| 14980.m02824 | Bm1_46495 | Zinc finger, C2H2 type | 14980 | 956839 | 950859 |
| | | family protein | | | |
| 14980.m02854 | Bm1_46520 | predicted protein | 14980 | 977886 | 975605 |
| 14981.m02374 | Bm1_46675 | membrane-associated | 14981 | 39898 | 37539 |
| | | RING-CH protein III, | | | |
| | | putative | | | |
| 14981.m02394 | Bm1_46775 | hypothetical protein | 14981 | 181813 | 180634 |
| 14981.m02398 | Bm1_46795 | protein T01B7.5, | 14981 | 197058 | 199888 |
| | | putative | | | |
| 14981.m02425 | Bm1_46940 | hypothetical protein | 14981 | 414288 | 412399 |
| 14981.m02431 | Bm1_46970 | hypothetical protein | 14981 | 433081 | 436430 |
| 14981.m02468 | Bm1_47145 | Cuticle collagen dpy-2 | 14981 | 618741 | 621413 |
| | | precursor, putative | | | |
| 14982.m02229 | Bm1_47280 | WD-repeat protein | 14982 | 145884 | 148988 |
| | | WDC146.-related | | | |
| 14982.m02233 | Bm1_47300 | conserved hypothetical | 14982 | 176677 | 178003 |
| | | protein | | | |
| 14982.m02242 | Bm1_47345 | 3 exoribonuclease | 14982 | 224027 | 223358 |
| | | family, domain 2 | | | |
| | | containing protein | | | |
| 14988.m00035 | Bm1_47525 | LD18634p-related | 14988 | 316 | 1513 |
| 14990.m07639 | Bm1_47600 | Suppressor of lurcher | 14990 | 133342 | 131804 |
| | | protein 1 precursor, | | | |
| | | putative | | | |
| 14990.m07735 | Bm1_48075 | M-phase phosphoprotein | 14990 | 791528 | 789719 |
| | | 6.-related | | | |
| 14990.m07789 | Bm1_48330 | Dihydrofolate | 14990 | 1122208 | 1122372 |
| | | reductase.-related | | | |
| 14990.m07802 | Bm1_48395 | hypothetical protein | 14990 | 1223422 | 1230844 |
| 14990.m07875 | Bm1_48760 | putative transcription | 14990 | 1665855 | 1666819 |
| | | factor | | | |
| 14990.m07934 | Bm1_49050 | hypothetical protein | 14990 | 1994394 | 1992848 |
| 14990.m07962 | Bm1_49180 | NADH-ubiquinone | 14990 | 2154157 | 2154862 |
| | | oxidoreductase 15 kDa | | | |
| | | subunit.-related | | | |
| 14990.m07974 | Bm1_49240 | hypothetical protein | 14990 | 2235255 | 2234454 |
| 14990.m07993 | Bm1_49335 | RUN domain containing | 14990 | 2327353 | 2321510 |
| | | protein | | | |
| 14990.m08056 | Bm1_49645 | Microcephalin., putative | 14990 | 2694625 | 2699652 |
| 14990.m08061 | Bm1_49670 | zgc: 101594-related | 14990 | 2722366 | 2723619 |
| 14990.m08076 | Bm1_49750 | ORF; putative | 14990 | 2844297 | 2845746 |
| 14990.m08080 | Bm1_49770 | Mitochondrial ribosomal | 14990 | 2859183 | 2860321 |
| | | protein L51/S25/CI- | | | |
| | | B8 domain containing | | | |
| | | protein | | | |
| 14990.m08109 | Bm1_49915 | conserved hypothetical | 14990 | 3031369 | 3028987 |
| | | protein | | | |
| 14990.m08113 | Bm1_49935 | Zn-finger in Ran binding | 14990 | 3063258 | 3068759 |
| | | protein and others | | | |
| | | containing protein | | | |
| 14990.m08128 | Bm1_50000 | Conserved hypothetical | 14990 | 3150735 | 3149495 |
| | | protein, putative | | | |
| 14990.m08152 | Bm1_50115 | Hypothetical UPF0172 | 14990 | 3367925 | 3366463 |
| | | protein CG3501.-related | | | |
| 14992.m10844 | Bm1_50365 | conserved hypothetical | 14992 | 261445 | 264437 |
| | | protein | | | |
| 14992.m10852 | Bm1_50395 | hypothetical protein | 14992 | 297686 | 301543 |
| 14992.m10900 | Bm1_50630 | hypothetical protein | 14992 | 565049 | 569020 |
| 14992.m10916 | Bm1_50710 | hypothetical protein | 14992 | 633755 | 635590 |
| 14992.m10933 | Bm1_50790 | hypothetical protein | 14992 | 750415 | 756643 |
| 14992.m10940 | Bm1_50825 | hypothetical protein | 14992 | 813901 | 815576 |
| 14992.m10976 | Bm1_51010 | hypothetical protein | 14992 | 1084073 | 1081005 |
| 14992.m10983 | Bm1_51095 | hypothetical protein | 14992 | 1120507 | 1121506 |
| 14992.m10992 | Bm1_51095 | Innexin inx-10, putative | 14992 | 1168627 | 1173698 |
| 14992.m11085 | Bm1_51520 | hypothetical protein | 14992 | 1810026 | 1811523 |
| 14992.m11124 | Bm1_51715 | hypothetical protein | 14992 | 2031741 | 2024616 |
| 14992.m11181 | Bm1_51995 | LBP/BPI/CETP family, | 14992 | 2419876 | 2416185 |
| | | C-terminal domain | | | |
| | | containing protein | | | |
| 14992.m11195 | Bm1_52065 | hypothetical protein | 14992 | 2529293 | 2532954 |
| 14992.m11236 | Bm1_52255 | TB2/DP1, HVA22 family | 14992 | 2821338 | 2824401 |
| | | protein | | | |
| 14992.m11262 | Bm1_52385 | hypothetical protein | 14992 | 3033019 | 3027189 |
| 14992.m11279 | Bm1_52475 | hypothetical protein | 14992 | 3152405 | 3147920 |
| 14992.m11295 | Bm1_52560 | hypothetical protein | 14992 | 3250873 | 3250144 |
| 14992.m11302 | Bm1_52595 | Ground-like domain | 14992 | 3284234 | 3282575 |
| | | containing protein | | | |
| 14992.m11304 | Bm1_52605 | hypothetical protein | 14992 | 3291373 | 3294207 |
| 14992.m11309 | Bm1_52630 | Lipase family protein | 14992 | 3332305 | 3334702 |
| 14992.m11311 | Bm1_52640 | hypothetical protein | 14992 | 3341057 | 3341888 |
| 14992.m11327 | Bm1_52720 | Conserved hypothetical | 14992 | 3436867 | 3435529 |
| | | protein, putative | | | |
| 14992.m11343 | Bm1_52795 | hypothetical protein | 14992 | 3541900 | 3543185 |
| 14992.m11347 | Bm1_52815 | conserved hypothetical | 14992 | 3560316 | 3556758 |
| | | protein | | | |
| 15009.m00162 | Bm1_53230 | hypothetical protein | 15009 | 22811 | 24399 |
| 15009.m00163 | Bm1_53210 | predicted protein | 15009 | | |
| 15036.m00014 | Bm1_53390 | Ubiquinone biosynthesis | 15036 | 2512 | 2359 |
| | | protein COQ4 homolog, | | | |
| | | putative | | | |
| 15076.m00116 | Bm1_53630 | hypothetical protein | 15076 | 9415 | 8682 |
| 15081.m00161 | Bm1_53755 | NADH-ubiquinone | 15081 | 32119 | 31463 |
| | | oxidoreductase subunit | | | |
| | | B14.5b.-related | | | |
| 15131.m00092 | Bm1_54105 | hypothetical protein | 15131 | 14381 | 9128 |
| 15131.m00094 | Bm1_54115 | RH01479p-related | 15131 | 22598 | 21749 |
| 15133.m00185 | Bm1_54140 | GH14561p-related | 15133 | 4090 | 4396 |
| 15135.m00169 | Bm1_54230 | zgc: 101038 protein- | 15135 | 24439 | 23471 |
| | | related | | | |
| 15161.m00145 | Bm1_54490 | hypothetical protein | 15161 | 44043 | 46596 |
| 15182.m00047 | Bm1_54705 | Nematode cuticle | 15182 | 5679 | 4458 |
| | | collagen N-terminal | | | |
| | | domain containing | | | |
| | | protein | | | |
| 15201.m00015 | Bm1_54895 | hypothetical protein | 15201 | 333 | 1378 |
| 15215.m00045 | Bm1_55030 | hypothetical protein | 15215 | 6517 | 8010 |
| 15256.m00006 | Bm1_55375 | carbamoyl-phosphate | 15256 | 1399 | 1563 |
| | | synthase, large subunit, | | | |
| | | putative | | | |
| 15258.m00019 | Bm1_55395 | Conserved hypothetical | 15258 | 3252 | 4478 |
| | | protein, putative | | | |
| 15285.m00017 | Bm1_55635 | hypothetical protein | 15285 | 8941 | 13192 |
| 15295.m00028 | Bm1_55690 | NADH-ubiquinone | 15295 | 8747 | 9561 |
| | | oxidoreductase B12 | | | |
| | | subunit.-related | | | |
| 15304.m00109 | Bm1_55745 | sulfakinin receptor | 15304 | 5742 | 8999 |
| | | protein, putative | | | |
| 15304.m00111 | Bm1_55755 | major sperm protein, | 15304 | 14901 | 15446 |
| | | putative | | | |
| 15304.m00118 | Bm1_55790 | hypothetical protein | 15304 | 39132 | 36587 |
| 15304.m00121 | Bm1_55805 | hypothetical protein | 15304 | 50477 | 53422 |
| 15304.m00123 | Bm1_55755 | major sperm protein, | 15304 | 14901 | 15446 |
| | | putative | | | |
| 15330.m00019 | Bm1_56035 | hypothetical protein | 15330 | 5121 | 1685 |
| 15344.m00010 | Bm1_56145 | conserved hypothetical | 15344 | 213 | 4892 |
| | | protein | | | |
| 15360.m00009 | Bm1_56230 | GH25683p, putative | 15360 | 323 | 1602 |
| 15384.m00013 | Bm1_56405 | hypothetical protein | 15384 | 2008 | 3195 |
| 15404.m00031 | Bm1_56515 | Nematode cuticle | 15404 | 3134 | 1277 |
| | | collagen N-terminal | | | |
| | | domain containing | | | |
| | | protein | | | |
| 15406.m00015 | Bm1_56530 | 28S ribosomal protein | 15406 | 154 | 2845 |
| | | S30, mitochondrial, | | | |
| | | putative | | | |
| 15418.m00009 | Bm1_56600 | cystatin-type cysteine | 15418 | 780 | 2220 |
| | | proteinase inhibitor CPI- | | | |
| | | 2, putative | | | |
| 15425.m00019 | Bm1_56645 | Hypothetical 19.4 kDa | 15425 | 5850 | 4756 |
| | | protein T09A5.5 in | | | |
| | | chromosome III, | | | |
| | | putative | | | |
| 15443.m00042 | Bm1_56805 | 50S ribosomal protein | 15443 | 7638 | 8916 |
| | | L10.-related | | | |
| 15452.m00015 | Bm1_56880 | hypothetical protein | 15452 | 1875 | 2025 |
| 15458.m00030 | Bm1_56915 | conserved hypothetical | 15458 | 124 | 568 |
| | | protein | | | |
| 15527.m00010 | Bm1_57420 | Zinc finger, C2H2 type | 15527 | 2297 | 3106 |
| | | family protein | | | |
| 15530.m00010 | Bm1_57435 | ELM2 domain containing | 15530 | 1682 | 354 |
| | | protein | | | |
| 15559.m00008 | Bm1_57645 | conserved hypothetical | 15559 | 2901 | 201 |
| | | protein | | | |
| 12479.m00026 | Bm1_00645 | Transmembrane cell | 12479 | 11016 | 9951 |
| | | adhesion receptor mua-3 | | | |
| | | precursor, putative | | | |
| 12584.m00064 | Bm1_01560 | conserved hypothetical | 12584 | 8270 | 6360 |
| | | protein | | | |
| 12621.m00166 | Bm1_01990 | chitin synthase 2 (chs-2) | 12621 | 185 | 1308 |
| | | fragment | | | |
| 12631.m00043 | Bm1_02065 | Abnormal cell migration | 12631 | 4978 | 1152 |
| | | protein 10, putative | | | |
| 12673.m00009 | Bm1_02325 | Immunoglobulin I-set | 12673 | 3787 | 1197 |
| | | domain containing | | | |
| | | protein | | | |
| 12849.m00036 | | Kunitz/Bovine | 12849 | 3944 | 4416 |
| | | pancreatic | | | |
| | | trypsin | | | |
| | | inhibitor | | | |
| | | domain | | | |
| | | containing | | | |
| | | protein | | | |
| 13204.m00045 | Bm1_05890 | B20-1 protein, putative | 13204 | 323 | 1656 |
| 13207.m00045 | Bm1_05925 | EGF-like domain | 13207 | 5569 | 2854 |
| | | containing protein | | | |
| 13207.m00046 | Bm1_05930 | Muscle positioning | 13207 | 12373 | 8852 |
| | | protein 4, putative | | | |
| 13210.m00167 | | Patched | 13210 | 53524 | 52734 |
| | | related family | | | |
| | | protein 12, | | | |
| | | isoform b, | | | |
| | | putative | | | |
| 13236.m00035 | Bm1_06130 | Nuclear anchorage | 13236 | 13917 | 11658 |
| | | protein 1-related | | | |
| 13271.m00048 | Bm1_06810 | Cuticle collagen dpy-7 | 13271 | 12722 | 10083 |
| | | precursor, putative | | | |
| 13456.m00013 | Bm1_09195 | Thyroglobulin type-1 | 13456 | 4146 | 6135 |
| | | repeat family protein | | | |
| 13480.m00175 | Bm1_09565 | Transmembrane amino | 13480 | 44824 | 50233 |
| | | acid transporter protein | | | |
| 13488.m00014 | Bm1_09610 | Troponin T, putative | 13488 | 2354 | 177 |
| 13617.m00050 | Bm1_10505 | cuticle collagen 34, | 13617 | 17986 | 20127 |
| | | putative | | | |
| 13761.m00027 | Bm1_11715 | Phospholipase c like | 13761 | 1332 | 309 |
| | | protein 1, isoform b, | | | |
| | | putative | | | |
| 13832.m00022 | Bm1_12300 | Fibronectin type III | 13832 | 649 | 2354 |
| | | domain containing | | | |
| | | protein | | | |
| 13879.m00034 | Bm1_12525 | RNA dependent RNA | 13879 | 9524 | 10985 |
| | | polymerase family | | | |
| | | protein | | | |
| 13894.m00035 | Bm1_12585 | small heat shock protein | 13894 | 2042 | 2542 |
| | | 12.6, putative | | | |
| 13939.m00062 | Bm1_13015 | Nematode cuticle | 13939 | 14226 | 12675 |
| | | collagen N-terminal | | | |
| | | domain containing | | | |
| | | protein | | | |
| 14134.m00015 | Bm1_15075 | PDZ domain containing | 14134 | 440 | 3980 |
| | | protein | | | |
| 14280.m00063 | Bm1_17130 | Kunitz/Bovine pancreatic | 14280 | 11169 | 10679 |
| | | trypsin inhibitor domain | | | |
| | | containing protein | | | |
| 14288.m00017 | Bm1_17360 | conserved hypothetical | 14288 | 2832 | 11 |
| | | protein | | | |
| 14289.m00013 | Bm1_17365 | RNA-directed RNA | 14289 | 2521 | 302 |
| | | polymerase 1-related | | | |
| 14332.m00026 | Bm1_18000 | Zinc finger, C2H2 type | 14332 | 2391 | 5302 |
| | | family protein | | | |
| 14356.m00524 | Bm1_18340 | LIN-7, putative | 14356 | 72258 | 71594 |
| 14383.m00056 | Bm1_18740 | Transmembrane cell | 14383 | 6819 | 8155 |
| | | adhesion receptor mua-3 | | | |
| | | precursor, putative | | | |
| 14456.m00009 | Bm1_19740 | Hypothetical protein | 14456 | 862 | 1068 |
| 14479.m00133 | Bm1_19990 | cuticle collagen 2 | 14479 | 16244 | 19999 |
| | | precursor, putative | | | |
| 14568.m00086 | Bm1_21210 | Innexin inx-14.-related | 14568 | 4954 | 6184 |
| 14590.m00344 | Bm1_21610 | PDZ domain containing | 14590 | 38866 | 40361 |
| | | protein | | | |
| 14629.m00079 | Bm1_22470 | conserved hypothetical | 14629 | 8429 | 4911 |
| | | protein | | | |
| 14770.m00166 | Bm1_25645 | hypothetical protein | 14770 | 16479 | 16947 |
| 14789.m00059 | Bm1_26235 | hypothetical protein | 14789 | 8913 | 10868 |
| 14804.m00022 | Bm1_26400 | Fibronectin type III | 14804 | 4058 | 20 |
| | | domain containing | | | |
| | | protein | | | |
| 14847.m00069 | Bm1_26685 | F25C8.3 protein-related | 14847 | 11682 | 12570 |
| 14899.m00015 | Bm1_27205 | hypothetical protein | 14899 | 2701 | 225 |
| 14907.m00570 | Bm1_27360 | hypothetical protein | 14907 | 67058 | 69505 |
| 14907.m00591 | Bm1_27330 | conserved hypothetical | 14907 | 53267 | 52044 |
| | | protein | | | |
| 14916.m00488 | Bm1_27570 | ARID/BRIGHT DNA | 14916 | 120637 | 119811 |
| | | binding domain | | | |
| | | containing protein | | | |
| 14926.m00034 | Bm1_28210 | hypothetical protein | 14926 | 21396 | 19323 |
| 14956.m00537 | Bm1_31780 | Clc-4 protein., putative | 14956 | 366586 | 364379 |
| 14961.m04954 | Bm1_32310 | Innexin family protein | 14961 | 418907 | 422111 |
| 14961.m05117 | Bm1_33125 | conserved hypothetical | 14961 | 1460575 | 1457288 |
| | | protein | | | |
| 14961.m05334 | Bm1_34195 | Filamin/ABP280 repeat | 14961 | 2826597 | 2815179 |
| | | family protein | | | |
| 14961.m05355 | Bm1_34300 | Nematode cuticle | 14961 | 2964725 | 2966387 |
| | | collagen N-terminal | | | |
| | | domain containing | | | |
| | | protein | | | |
| 14965.m00415 | Bm1_34840 | collagen col-34- | 14965 | 128392 | 130818 |
| | | Caenorhabditis elegans, | | | |
| | | putative | | | |
| 14968.m01485 | Bm1_35480 | hypothetical protein | 14968 | 189559 | 191759 |
| 14972.m07061 | Bm1_36850 | hypothetical protein | 14972 | 690327 | 686987 |
| 14972.m07168 | Bm1_37370 | conserved hypothetical | 14972 | 1387688 | 1395188 |
| | | protein | | | |
| 14972.m07534 | Bm1_39165 | cAMP-dependent protein | 14972 | 4167551 | 4167742 |
| | | kinase regulatory chain, | | | |
| | | putative | | | |
| 14972.m07898 | Bm1_40905 | Putative glutamate | 14972 | 6509279 | 6512457 |
| | | synthase, putative | | | |
| 14972.m07904 | Bm1_37335 | ankyrin-related unc-44- | 14972 | 1370164 | 1371628 |
| | | related | | | |
| 14973.m02693 | Bm1_41500 | conserved hypothetical | 14973 | 730367 | 737643 |
| | | protein | | | |
| 14975.m04416 | Bm1_42395 | Nematode cuticle | 14975 | 626425 | 628055 |
| | | collagen N-terminal | | | |
| | | domain containing | | | |
| | | protein | | | |
| 14975.m04428 | | Patched | 14975 | 708159 | 704998 |
| | | family protein | | | |
| 14975.m04547 | Bm1_43030 | protein C18H9.7, | 14975 | 1356110 | 1354082 |
| | | putative | | | |
| 14977.m04877 | Bm1_43170 | Transmembrane amino | 14977 | 190435 | 186932 |
| | | acid transporter protein | | | |
| 14977.m04993 | Bm1_43725 | Mov34/MPN/PAD-1 | 14977 | 936281 | 935192 |
| | | family protein | | | |
| 14977.m04996 | Bm1_43740 | conserved hypothetical | 14977 | 960724 | 956647 |
| | | protein | | | |
| 14980.m02767 | Bm1_46225 | hypothetical protein | 14980 | 531468 | 532992 |
| 14981.m02440 | | hypothetical | 14981 | 468578 | 467689 |
| | | protein | | | |
| 14990.m07640 | Bm1_47605 | CUB domain containing | 14990 | 136663 | 135121 |
| | | protein | | | |
| 14990.m07659 | Bm1_47700 | Mitochondrial import | 14990 | 232655 | 234681 |
| | | inner membrane | | | |
| | | translocase subunit | | | |
| | | Tim17 family protein | | | |
| 14990.m07706 | Bm1_47930 | VAB-10A protein-related | 14990 | 571636 | 570404 |
| 14990.m07759 | Bm1_48195 | Innexin family protein | 14990 | 931282 | 934769 |
| 14992.m10954 | Bm1_50900 | Conserved hypothetical | 14992 | 926641 | 931225 |
| | | protein, putative | | | |
| 14992.m11025 | Bm1_51260 | Innexin family protein | 14992 | 1399989 | 1406584 |
| 14992.m11129 | Bm1_51735 | Troponin I, putative | 14992 | 2080336 | 2083277 |
| 14992.m11174 | Bm1_51960 | conserved hypothetical | 14992 | 2373772 | 2376231 |
| | | protein | | | |
| 14992.m11212 | Bm1_52140 | P40-related | 14992 | 2662635 | 2659469 |
| 15002.m00035 | Bm1_53115 | hypothetical protein | 15002 | 1933 | 291 |
| 15009.m00158 | Bm1_53205 | hypothetical protein | 15009 | 272 | 6328 |
| 15059.m00090 | Bm1_53505 | myotactin form B, | 15059 | 8632 | 410 |
| | | putative | | | |
| 15059.m00092 | Bm1_53515 | conserved hypothetical | 15059 | 32453 | 29060 |
| | | protein | | | |
| 15081.m00162 | Bm1_53760 | cytochrome-c oxidase, | 15081 | 32931 | 32512 |
| | | putative | | | |
| 15121.m00020 | Bm1_54070 | F25C8.3 protein-related | 15121 | 2385 | 411 |
| 15165.m00050 | Bm1_54575 | Membrane calcium | 15165 | 29090 | 31398 |
| | | atpase protein 3, | | | |
| | | isoform a, putative | | | |
| 15213.m00007 | Bm1_55000 | Troponin T, putative | 15213 | 2590 | 1031 |
| 15253.m00027 | Bm1_55355 | Protein FAM34A.-related | 15253 | 8478 | 10223 |
| 15297.m00023 | Bm1_55705 | Conserved hypothetical | 15297 | 208 | 1397 |
| | | protein, putative | | | |
| 15322.m00022 | Bm1_55970 | hypothetical protein | 15322 | 6778 | 4783 |
| 15331.m00008 | Bm1_56045 | myosin-like protein, | 15331 | 2131 | 11 |
| | | putative | | | |
| 15370.m00010 | Bm1_56290 | ARID/BRIGHT DNA | 15370 | 3047 | 65 |
| | | binding domain | | | |
| | | containing protein | | | |
| 15398.m00011 | Bm1_56480 | RhoGAP domain | 15398 | 3587 | 9833 |
| | | containing protein | | | |
| 15487.m00008 | Bm1_57145 | Fibronectin type III | 15487 | 40 | 2883 |
| | | domain containing | | | |
| | | protein | | | |
| 15560.m00010 | Bm1_57650 | conserved hypothetical | 15560 | 2283 | 85 |
| | | protein | | | |

What is claimed:

1. A method for selecting drug targets, comprising:
(a) identifying one or more essential or functionally important sequences from a model organism using pre-existing genomic and phenotypic data.
(b) comparing sequences from (a) with a DNA or peptide sequence from a pathogen to store homologous sequences;
(c) comparing sequences from the pathogen with the DNA or peptide sequence from a host organism to store those sequences absent in the host organism; and
(d) comparing sequences from (b) and (c) to identify shared sequences, the shared sequences being a drug target.

2. A method according to claim 1, wherein two or more of steps (a)-(d) are performed sequentially.

3. A method according to claim 1, wherein two or more of steps (a)-(d) are performed in parallel.

4. A drug target, comprising a DNA sequence or protein selected from Table 1.

5. A computer-based system for identifying drug targets comprising:
(a) a memory for storing a plurality of databases, wherein the plurality of databases comprise a plurality of sequence databases;
(b) a processor in communication with the memory; and
(c) an output device in communication with the processor, wherein the processor is configured to:
(i) group sequences belonging to a model organism, a pathogen and a host from one or more of the plurality of sequence databases;
(ii) identify essential or functionally important sequences from the model organism by matching phenotypic data with sequences of the model organism grouped in (i);
(iii) compare sequences of the pathogen with sequences identified in (ii);
(iv) compare sequences of the pathogen with the host sequences and select pathogen sequences that do not share sequence similarity with the host sequences;
(v) compare sequences from (iii) and (iv) to identify sequences corresponding to sequences encoding drug targets; and
(vi) cause at least some of the sequences identified as drug targets in (v) to be displayed on the output device.
